(19) United States
(12) Patent Application Publication
(10) Pub. No.: US 2010/0204780 A1
(43) Pub. Date: Aug. 12, 2010

(54) FLEXIBLE EXTENDABLE STENT AND METHODS OF SURFACE MODIFICATION THEREFOR

(75) Inventors: Thilo U. Fliedner, Niederpocking (DE); Eric S. Ryan, Hopkinton, MA (US)

Correspondence Address: MILLS & ONELLO LLP, ELEVEN BEACON STREET, SUITE 605, BOSTON, MA, 02108, US

(73) Assignee: CorNova, Inc., Burlington, MA (US)

(21) Appl. No.: 12/679,585

(22) PCT Filed: Sep. 26, 2008
(86) PCT No.: PCT/US08/77871

§ 371 Date: Mar. 23, 2010

Related U.S. Application Data
(60) Provisional application No. 60/975,383, filed on Sep. 26, 2007.
(60) Provisional application No. 61/013,246, filed on Dec. 12, 2007.

(51) Int. Cl. A61F 2/06; B05D 1/12

(52) U.S. Cl. 623/1.16; 427/2.25

(57) ABSTRACT

Stent strut and surface geometries are provided for enhancing surface coating applications while providing highly beneficial biomechanical properties. A low-profile, flexible, expandable, elongated, stent assembly is provided and defined by a structure of connected circumferential arrays of webs or bends, the webs or bends and their connections having limited degrees of curvature that help avoid interference during various surface-modifying and surface-enhancing processes.

### RELATED APPLICATIONS

This application is a continuation-in-part of U.S. patent application Ser. No. 11/613,443 filed on Dec. 20, 2006, which is a continuation-in-part of U.S. patent application Ser. No. 29/252,668 filed Jan. 25, 2006, issued as U.S. Patent No. D553,746 and U.S. application Ser. No. 29/252,669 filed Jan. 25, 2006, issued as U.S. Pat. No. D553,747, the contents of each of which are herein incorporated by reference in their entirety. This application is also a continuation-in-part of U.S. patent application Ser. No. 11/843,376 filed on Aug. 22, 2007, published on Jul. 24, 2008 as U.S. Patent Application Publication No. 2008-0177371-A1 and U.S. patent application Ser. No. 11/843,402 filed on Aug. 22, 2007, published on Sep. 4, 2008 as U.S. Patent Application Publication No. 2008-0215132-A1, the contents of each of which are herein incorporated by reference in their entirety. This application claims the benefit of U.S. Patent Application No. 61/013,246 filed on Dec. 12, 2007, and U.S. Patent Application No. 60/975,383 filed on Sep. 26, 2007, the contents of each of which are herein incorporated by reference in their entirety. This application is related to U.S. Patent Application No. 60/823,692 filed on Aug. 28, 2006, U.S. Patent Application No. 60/825,434 filed on Sep. 13, 2006, U.S. Patent Application No. 60/895,924 filed on Mar. 20, 2007, and U.S. Patent Application No. 60/941,813 filed on Jun. 4, 2007, the contents of each of which are herein incorporated by reference in their entirety.

### BACKGROUND OF THE INVENTION

1. Field of the Invention

Embodiments of the present invention relate to medical stents, which are implantable devices for propping open and maintaining the patency of vessels and ducts in the vasculature of a human being.

2. Description of the Related Art

Stents are implantable prosthesis used to maintain and/or reinforce vascular and endoluminal ducts in order to treat and/or prevent a variety of medical conditions. Typical uses include maintaining and supporting coronary arteries after they are opened and unclogged, such as through an angioplasty operation. A stent is typically deployed in an unexpanded or crimped state using a catheter and, after being properly positioned within a vessel, is then expanded into its final shape (such as with an expandable balloon incorporated into the catheter).

As a foreign object inserted into a vessel, a stent can potentially impede the flow of blood. This effect can also be exacerbated by the undesired growth of tissue and on and around the stent, potentially leading to complications including thrombosis and restenosis. Thus, stents are manufactured to minimize impedance of a vessel while being capable of maintaining their expanded state. Typical stents have the basic form of an open-ended tubular element supported by a mesh of thin struts with openings formed therein between. Designs typically include strong, flexible, and malleable base materials and, in order to resist excessive tissue growth, can include surface materials of greater biocompatibility and/or active anti-proliferative mechanisms such as drug-eluting polymers.

However, many commercially available coated stents suffer from problems including corrosion, flaking, cracking, and other strut and surface imperfections. The effects of flaking or cracking of surface materials, which create a less smooth surface and can also substantially negate anti-growth properties, may even cause a serious blockage resulting in death. Many of these problems arise because of the difficulty in effectively coating the thin, angular struts of a typical stent which must undergo flexing and deformation during deployment. A typical stent strut pattern is generally designed to minimize the level of stent-to-tissue contact, promote even expansion, and maintain sufficient retention force while avoiding such problems as foreshortening, or the shortening of the stent as it expands.

The resulting complex patterns that embody many stents thus often require complex, expensive coating and/or other surface modification mechanisms. Preferred techniques for coating/surfacing stents generally involve polishing, cleaning, and/or deposition processes such as, for example, electro-polishing, electrochemical deposition, ultrasonic spray systems and/or plasma-based coating systems. The level of angularity and irregularity of a stent pattern can significantly effect a surface modification process, and, in particular, the uniformity, adhesiveness, and thickness of a coating.

For example, an area of a stent strut pattern with sharply angular features may inordinately block some of a surface modification process, including a cleaning process, and further block a coating material from evenly collecting and adhering along these features. When a spraying or bombardment type of process is employed, the heavy angularity and irregularity of the surface makes uniformly targeting the irregularly featured and/or curved surfaces highly challenging.

Furthermore, many surface modification and coating processes involve a charged target substrate which operates to attract adhesion/density enhancing bombardment and/or deposition of metallic and/or charged particles. If an electrically charged substrate, for example, has a portion with tight curvature (or a decreased radius of curvature), the resulting magnetic field along that portion of the substrate will tend to cancel out within the immediate area of curvature, reducing the potential and effect of the surface modification/coating process in these areas. The impairment of a coating process in these areas may necessitate adding more coating overall to the entire stent surface in order to accommodate a sufficiently extensive coating. The increased thickness of a coating can reduce the flexibility of the stent and/or increase the likelihood of cracking.

Another complication that can occur in areas of sharp angularity is “webbing,” where areas between closely spaced surfaces can essentially be filled in with material, causing the coating to split and/or flake when the area opens during expansion of the stent. Furthermore, these areas of highly angular and/or irregular shapes can be inherently more susceptible to cracking with or without coatings due to the stresses they undergo when flexing occurs during expansion.

Thus, there is a need for stents which have both the preferred bio-mechanical properties and that are formed to provide optimal surfaces for both biocompatible and bio-active coatings.

### SUMMARY OF THE INVENTION

Embodiments of the present invention relate to medical stent assemblies comprised of elongated tubular patterns of metal capable of expanding and propping open a vessel or duct within a living, human being. It is one object of the present invention to provide a substrate structure with curvilinear features optimized for providing excellent bio-mechanical properties (e.g. even expansion, retention force, flexibility, strength, avoidance of foreshortening) in acting as a vessel prosthesis while permitting the application of relatively thin, smooth, and/or even surface-enhancing coatings. Embodiments of the invention can provide particularly optimal surfaces for coating applications involving the use of spraying or bombarding particles about the substrate structure such as with, for example, ion-assisted deposition.

An aspect of the present invention comprises a stent having struts forming a plurality of connected circumferential arrays of curves or bends, the curves or bends and their connections having radii of curvature of at least about 65 micrometers. In an embodiment, the curves or bends and their connections have radii of curvature of at least about 80 micrometers.

In an embodiment, the stent is coated with a surface-modifying material. In an embodiment, the surface-modifying material is applied with a surface modification process employing the aid of a magnetic bias along the stent substrate for attracting the surface-modifying material. In an embodiment, the surface modification process includes charging the stent so as to produce the magnetic bias.

In an embodiment, the surface modification process includes at least one of electrochemical deposition, electroplating, electro-polishing, ion-bombardment, and ion-assisted deposition.

In an aspect of the invention, a flexible, expandable, elongated, stent assembly comprises a generally cylindrically-shaped channel that extends along a longitudinal axis and further comprises a plurality of openings in the channel. The openings are defined by a structure of connected circumferential arrays of webs or bends, wherein, in an unexpanded state of the stent assembly, the webs or bends and their connections have minimum radii of curvature of at least about 65 microns.

In an embodiment, the webs or bends and their connections have minimum radii of curvature of at least about 80 microns.

In an embodiment, each of the circumferential arrays of webs or bends comprises a first pattern of lengthwise-sized bends and a second pattern of lengthwise elongatedly-sized bends positioned at regular intervals on each circumferential array.

In an embodiment, the webs or bends are in a switchback configuration and are substantially smoothly arcuately-shaped. In an embodiment, a substantial portion of each of said arcuately-shaped, generally hairpin-like curved webs or bends form arcs of generally the same orientation with respect to the circumference of said stent assembly.

In an embodiment, each of the circumferential arrays are connected to one another by two or more crosslinks.

In an embodiment, each of the two or more crosslinks extend between lengthwise-elongated sized bends of said adjacent arrays.

In an embodiment, each of the two or more crosslinks extending from a circumferential array is substantially circumferentially offset from every crosslink extending from an opposite side of the same circumferential array.

In an embodiment, each crosslink extending from one side of each circumferential array is circumferentially offset by at least about 60 degrees from every crosslink extending from an opposite side of the same circumferential array. In an embodiment, each crosslink extending from one side of a circumferential array is circumferentially offset by at least about 90 degrees from every crosslink extending from an opposite side of the same circumferential array.

In an embodiment, each of the two or more crosslinks is arc-shaped and circumferentially oriented in a direction similar to each of the circumferential arrays of webs or bends.

In an embodiment, the assembly has one or more surface layers thereon. In an embodiment, the one or more surface layers comprises a metal capping layer comprising a predominant proportion of a substantially biocompatible metal. In an embodiment, the substantially biocompatible metal comprises at least one of platinum, platinum-iridium, tantalum, titanium, tin, indium, palladium, gold and alloys thereof. In an embodiment, the metal capping layer consists essentially of pure platinum.

In an embodiment, the one or more surface layers further comprises an adhesion layer positioned between the substrate and the metal capping layer. In an embodiment, the adhesion layer comprises a predominant proportion of palladium.

In an embodiment, the metal capping layer and all surface layers within the metal capping layer have a total thickness of less than or equal to about 0.5 microns. In an embodiment, the metal capping layer and all surface layers within the metal capping layer have a total thickness of less than about 0.25 microns. In an embodiment, the metal capping layer has a density of greater than about 95% full bulk density.

In an embodiment, the one or more surface layers comprises a polymer.

In an embodiment, external surfaces of the webs or bends and cross-links are separated from opposing external surfaces of the webs or bends along normal straight-line spans by a minimum of about 130 microns.

In another aspect of the invention, a flexible, expandable, elongated stent assembly comprises a generally cylindrically-shaped channel that extends along a longitudinal axis, and further comprises a plurality of openings in said channel. The openings are defined by a substrate structure of webs or bends, the webs or bends having minimum radii of curvature of at least about 65 micrometers. The stent assembly further comprises one or more surface layers over the substrate structure of webs or bends.

In an embodiment, the one or more surface layers comprises a metal capping layer with a predominant proportion of a substantially biocompatible metal.

In an embodiment, the substantially biocompatible metal is platinum. In an embodiment, the metal capping layer consists essentially of pure platinum.

In an embodiment, the combined thickness of the metal capping layer and all surface layers within the metal capping layer is less than about 0.5 microns.

In an embodiment, the combined thickness of the metal capping layer and all surface layers within the metal capping layer is less than about 0.25 microns.

In an embodiment, external surfaces of the webs or bends are separated from opposing external surfaces of the webs or bends along normal straight-line paths by at least about 130 microns.

In another aspect of the invention, a flexible, expandable, elongated stent assembly comprises a generally cylindrically-shaped channel that extends along a longitudinal axis, and further comprises a plurality of openings therein, the openings being defined by a substrate of substantially smoothly and arcuately-shaped webs or bends. External surfaces of the webs or bends are separated from opposing external surfaces of the webs or bends along normal straight-line paths by at least about 130 microns.

In an embodiment, wherein external surfaces of the webs or bends are separated from opposing external surfaces of the webs or bends along normal straight-line paths by at least about 160 microns.

In an embodiment, there are one or more surface layers on the webs or bends.

In another aspect of the invention, a flexible, expandable, elongated stent assembly comprises a generally cylindrically-shaped channel that extends along a longitudinal axis, and further comprises a plurality of openings therein. The openings are defined by a structure of connected circumferential arrays of webs or bends, wherein, in an unexpanded state of the stent assembly, the webs or bends and their connections have minimum radii of greater than about 50 microns. There are one or more surface layers on the stent assembly.

In an embodiment, the one or more surface layers comprises a metal capping layer of predominantly platinum. In an embodiment, the metal capping layer comprises a predominant proportion of platinum. In another embodiment, the metal capping layer consists essentially of platinum.

In an embodiment, the essentially platinum capping layer and all surface layers within the essentially platinum capping layer have a combined thickness of less than about 15,000 angstroms. In an embodiment, the essentially platinum capping layer and all surface layers within the essentially platinum capping layer have a combined thickness of between about 100 and 5000 angstroms.

In an aspect of the invention, a method of coating a flexible, expandable stent assembly is provided, the method including providing a stent including a generally cylindrically-shaped channel having a longitudinal axis and having a plurality of openings therein. The openings are defined by a substrate structure of webs or bends, the webs or bends having minimum radii of curvature of at least about 65 micrometers. The method further includes directing at least one stream of coating particles toward the substrate structure so as to form one or more layers of coating particles over the substrate.

In an embodiment, the webs or bends have minimum radii of curvature of at least about 80 microns.

In an embodiment, directing at least one stream of coating particles toward the substrate includes the use of at least one of electrochemical deposition, electroplating, electro-polishing, and ion-assisted deposition.

In an embodiment, directing the at least one stream of coating particles includes an ion-assisted deposition process including simultaneously directing the coating particles and bombarding ions toward the substrate in a substantially collinear manner.

In an embodiment, directing the at least one stream of coating particles toward the substrate includes forming a metal capping layer over the substrate, the metal capping layer including a predominant proportion of a highly biocompatible metal.

In an embodiment, the highly biocompatible metal is at least one of platinum, platinum-iridium, tantalum, titanium, tin, indium, palladium, gold and alloys thereof. In an embodiment, the biocompatible metal consists essentially of platinum.

In an embodiment, the combined thickness of the metal capping layer and all layers within the metal capping layer is less than about a micron. In an embodiment, the metal capping layer and all surface layers within the metal capping layer is less than about 0.5 microns. In an embodiment, the combined thickness of the metal capping layer and all surface layers within the metal capping layer is less than about 0.25 microns.

In an embodiment, the at least one stream of coating particles comprises a stream of polymer material.

In an embodiment, each of the circumferential arrays of webs includes a first pattern of lengthwise-sized bends and a second pattern of lengthwise-elongatedly sized bends at regular intervals on each circumferential array.

In an embodiment, the webs or bends are generally hairpin-like and are substantially smoothly arcuately-shaped.

In an embodiment, each of the circumferential arrays are connected to one another by two or more cross-links.

In an embodiment, each of the two or more crosslinks are arcuately-shaped and extend between lengthwise-elongated sized bends of said adjacent arrays.

In an embodiment, the one or more layers of coating materials has a total thickness of equal to or less than about a micron.

In an embodiment, the one or more layers of coating materials has a total thickness of equal to or less than about 0.5 microns.

In an embodiment, the one or more layers of coating materials has a total thickness of less than about 0.25 microns.

In an embodiment, the webs or bends are separated from opposing portions along normal straight-line spans by a minimum of about 130 microns.

In an embodiment, a substantially uniform magnetic field is generated about the webs or bends while the at least one stream of coating particles is directed toward the substrate.

In an embodiment, the substantially uniform magnetic field is generated by providing a voltage across the webs or bends.

In an embodiment, the voltage across the webs or bends is actively applied to the webs or bends. In an embodiment, the voltage across the webs or bends is between about −20VDC and −1000VDC. In an embodiment, the voltage across the webs or bends is between about −20VDC and −100VDC.

In another aspect of the invention, a method of coating a flexible, expandable stent assembly comprises providing a stent comprising a generally cylindrically-shaped channel, having a longitudinal axis, and having a plurality of openings therein, said openings being defined by a substrate of circumferential arrays of webs or bends, two or more cross-links connecting adjacent circumferential arrays of said webs or bends, the webs or bends, cross-links and their connections having minimum radii of curvature of at least about 65 micrometers. The method further includes directing at least one stream of coating particles toward the substrate so as to form one or more layers of coating particles over the substrate.

In an embodiment, the webs or bends, cross-links and their connections have minimum radii of curvature of at least about 80 micrometers.

In an embodiment, directing the at least one stream of coating particles toward the substrate includes the use of ion-assisted deposition with at least one or more magnetrons.

In another aspect of the invention, a method of coating a flexible, expandable stent assembly comprises providing a stent having a generally cylindrically-shaped channel, having a longitudinal axis, and having a plurality of openings therein, said openings being defined by a substrate of webs or bends, the webs or bends separated from opposing portions along normal straight-line spans by a minimum of about 130 microns. The method further includes directing at least one stream of coating particles toward the substrate so as to form one or more layers of coating particles over the substrate.

In an embodiment, the webs or bends are separated from opposing portions along normal straight-line spans by a minimum of about 160 microns.

In another aspect of the invention, a method of coating a flexible, expandable stent assembly comprises providing a stent comprising a generally cylindrically-shaped channel, having a longitudinal axis, and having a plurality of openings therein, the openings being defined by a substrate of webs or bends, the webs or bends having minimum radii of curvature of greater than about 50 micrometers, and further comprises directing at least one stream of coating particles toward the substrate so as to faun one or more layers of coating particles over the substrate.

In an embodiment, directing at least one stream of coating particles includes an ion-assisted deposition process.

In an embodiment, the one or more layers of coating particles over the substrate includes an adhesion layer of predominantly palladium directly on the substrate and a metal capping layer of predominantly platinum over the adhesion layer.

In an embodiment, the one or more layers of coating particles includes a metal capping layer consisting essentially of platinum, in which the metal capping layer and all surface layers within the metal capping layer have a combined thickness of between about 100 and 5000 angstroms.

### BRIEF DESCRIPTION OF THE DRAWINGS

The objects and advantages of embodiments of the present invention will become more apparent when viewed in conjunction with the following drawings, in which:

FIG. 1A is a longitudinal presentation, in a flat or “planar” array, of a stent assembly according to embodiments of the invention;

FIG. 1B is an enlarged plan view of a portion of a circumferential array of arcuately-shaped hairpin-like bends of the stent assembly shown in FIG. 1A;

FIG. 2 is a side elevational view of a stent assembly in a cylindrical configuration according to embodiments of the invention; and,

FIG. 3 is a side-perspective illustrative schematic of an apparatus for coating an implantable device using multiple magnetrons according to embodiments of the invention.

### DETAILED DESCRIPTION OF EMBODIMENTS

The accompanying drawings are described below, in which example embodiments in accordance with the present invention are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that “adjacent” does not necessarily imply contact but may connote an absence of the same type of element(s) therein between “adjacent” elements.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

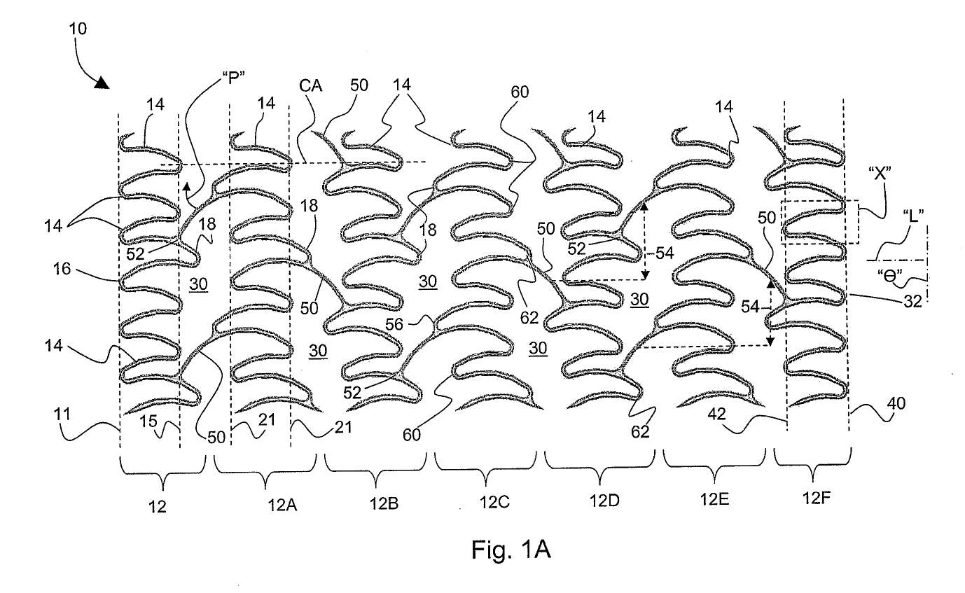

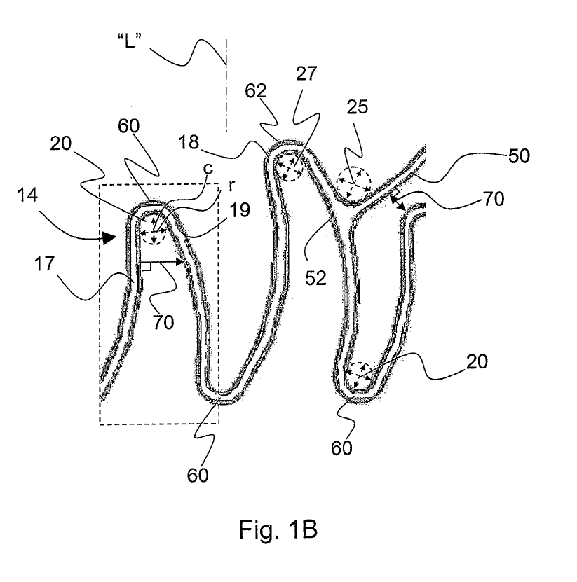

Referring now to the drawings in detail, and particularly to FIG. 1A, a stent assembly 10 in accordance with an embodiment of the present invention is represented in a flat or planar configuration for ease of understanding. The medical stent assembly 10 is comprised of an elongated tubular pattern of metal capable of expanding and propping open a vessel or duct within a living being, as represented in its cylindrical form, in FIGS. 1A and 1B. The stent assembly 10 comprises a plurality of web-like, circumferential arrays 12, 12A, . . . , 12F of bends or loops 14 that extend in a circumferential direction along “θ”. In an embodiment, a loop 14 in a circumferential array is in the configuration of an arcuately-shaped “hairpin-like” or “switchback” curve, as indicated within the dashed rectangle “X” shown in FIG. 1A.

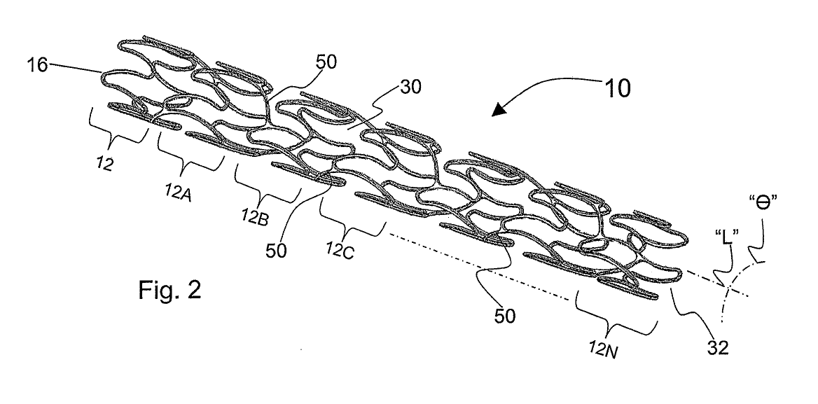

In an embodiment, the circumferential arrays 12, 12A, . . . , 12F of switchback loops or hairpin-like curves are each spaced apart from one another along the longitudinal axis “L” of the stent assembly 10, as shown in FIGS. 1A and 2. In an embodiment, each of the circumferential arrays 12-12F comprises a first pattern of lengthwise-size bends 16 and a second pattern of lengthwise elongatedly-sized bends 18 that are positioned at regular intervals on the circumferential array. In an embodiment, the loops or bends 14 at a first end 11 of the stent assembly 10 are all generally in peripheral alignment with one another, as indicated by their edges in alignment with the dashed line “11”. In an embodiment, the first or leftmost circumferential array 12 comprises a plurality of switchback or hairpin-like bends, curves, or loops 14, wherein every third switchback or hairpin-like curve or loop 14 is a lengthwise elongatedly-sized bend, curve or loop 18 positioned on the inwardly directed side of the first or leftmost circumferential array 12 that extends longitudinally beyond a peripheral border 15, while the remaining switchback or hairpin-like curves or loops 14 of the first or leftmost circumferential array 12 do not extend inwardly beyond the peripheral border 15. Further, the elongated loops 18 in each of the circumferential arrays 12, 12A . . . 12F comprising at least every third of the switchbacks or hairpin-like curves or loops 14 may extend longitudinally beyond one or more of their peripheral border alignments, as indicated by the dashed lines “21” and 15” of their adjacent bends, in an exemplary manner, for the two leftmost arrays 12 and 12A.

In an embodiment, a plurality of preferably smoothly curved, arcuate cross-links 50 are arranged so as to connect diagonally adjacent lengthwise elongatedly-sized loops 18 between longitudinally adjacent arrays 12, 12A etc., of bends or curves 14. Those elongated loops 18 preferably comprise every third loop 14 as most easily seen in FIG. 1A.

The second and successive circumferential arrays 12A, 12B etc, of switchback or hairpin-like curves or loops 14 are in generally corresponding longitudinal alignment with the switchback or hairpin-like curves or loops 14 of the first circumferential array 12 of loops 14 at the first end 16 of the stent assembly 10, as indicated by line CA, shown in FIG. 1A passing through the tips of the loops 14, which may be called “fronds” in keeping with a “Palm Tree” shape described herein in greater detail. That is, a switchback or loop 14 of an Nth circumferential array 12N of the plurality of circumferential arrays, for example, circumferential array 12D, is in generally longitudinal alignment with a corresponding switchback or loop 14 in a N+1 circumferential array 12N+1 of the plurality of circumferential arrays, for example, circumferential array 12E, of switchback or hairpin-like curves or loops 14. In another embodiment, the successive circumferential arrays 12A, 12B, . . . , 12F of loops 14 can be minimally longitudinally offset by a predetermined amount from the loops 14 of the first circumferential array 12.

In an embodiment, each adjacent circumferential array 12, 12A, . . . , 12F of loops or arcuately-shaped hairpin-like curves 14 is joined to its longitudinally adjacent circumferential array 12A, 12B, . . . , 12F of loops or hairpin-like curves 14 by at least two smoothly curved arcuate cross-links 50. Each cross-link 50 extends from a mid-portion 52 of a curved section of arch of an elongated switchback loop 18 to a tip 56 of the curved hairpin-like curve or bend 14 on a generally diagonally adjacent elongated curved switchback loop 18, as shown in FIGS. 1A and 1B. As a stent in accordance with this embodiment of the invention is expanded, e.g., with the use of an angioplasty balloon, the rotation or “pivoting” of a cross-link 50 pulls a curved section of an arch at a mid-portion 52 in both a circumferential direction (along “θ”) and a longitudinal direction (along “L”), thus distributing strut-to-tissue surface support of the circumferential array in both the circumferential and longitudinal directions. The circumferential pulling (or torque) of the cross-links during expansion on every other of the circumferential arrays (e.g., 12, 12B, and 12D) causes the circumferential arrays 12, 12A, 12B, . . . , 12F to shift circumferentially with respect to each adjacent circumferential array during expansion.

In accordance with various embodiments of the invention, the general patterns described herein can be adapted for differently sized stents or stents of different strengths varied according to need. For example, the frequency or number of circumferential arrays may be varied and the number of hairpin-like curves or loops may be varied as necessary for each circumferential array. For example, embodiments of the pattern with six hairpin-like loops for each circumferential array can provide for a stent length of about 9 mm with three columns of circumferential arrays, a length of about 12 mm with four columns of circumferential arrays, a length of about 15 mm with five columns of circumferential arrays, etc. These embodiments can have, for example, initial outer diameters of about 2 mm, crimped inner diameters of about 0.7 mm, and deployed outer diameters of about 2.75 mm, 3.0 mm, 3.5 mm, or 4.0 mm.

The elongated switchback loops 18 in each set of peripherally adjacent bends on adjacent circumferential arrays 12A etc. extend longitudinally beyond the bends or tips of their circumferentially adjacent hairpin-like curves 14, as indicated by the dashed lines 15, 21, and 42, shown in FIG. 1A.

In an embodiment, a generally semi-circumferentially extending annular, circumferentially elongated gap or space 30 is formed between adjacent arrays, for example, between array 12 and longitudinally adjacent array 12A, wherein the adjacent arrays defined by their respective circumferential loops 14 and the arcuate cross-links 50 resemble the aforementioned branched “palm tree” configuration, conspicuously shown at least in FIG. 1A.

The last circumferential array of switchback loops or hairpin-like curves 14 on the second end 32 of the stent assembly 10, for example, circumferential array 12F shown in FIG. 1A, has an edge array of bends 14 thereon which are in substantial peripheral alignment with one another, as indicated by their common alignment by a dashed line “40,” as shown in FIG. 1A. The last circumferential array 12F at the second end 32 of the stent assembly 10 also has elongated bends or elongated switchback loops 18, extending longitudinally beyond the peripheral edge of the adjacent switchback loops or hairpin-like curves 14 on that particular circumferential array 12F, for example, as indicated by their extending longitudinally “inwardly” beyond the dashed line 42.

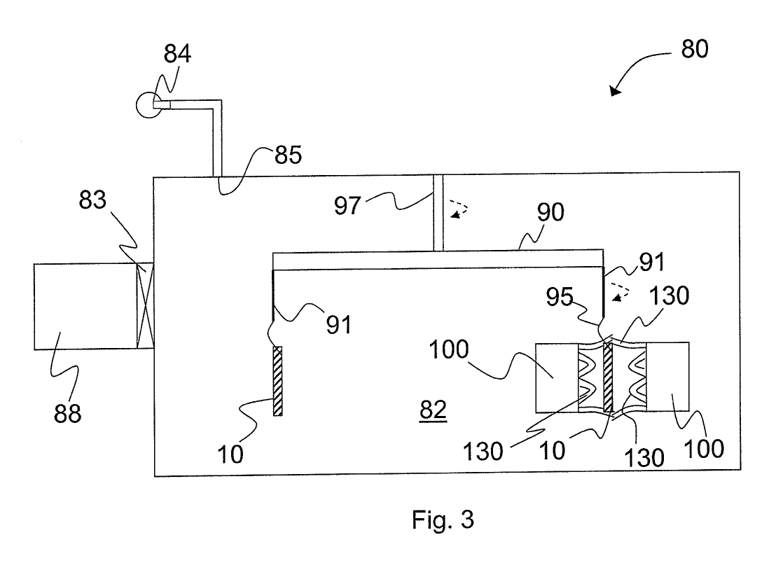

Thus, in an embodiment, a plurality of annular “palm-tree” shaped gaps 30 are formed between adjacent circumferential arrays 12, 12A etc. of switchback loops or hairpin-like curves 14 spans about 180 degrees of the circumference of the stent assembly 10 at that particular longitudinal location between adjacent arrays 12, 12A etc. The 180 degree clear, open, circumferentially disposed, “palm-tree” shaped “open cell” space 30 between adjacent circumferential arrays 12, 12A etc. generally comprises a “half periphery” of the stent assembly 10 which, as described hereinwith regard to FIG. 3, permits a second stent assembly (not shown) to be passed through and expand outwardly as in a vessel bifurcation, since the multiple longitudinally-dispersed, half-circumference “open cell” structure of each particular stent assembly 10 permits such multiple stent assembly interdigitation. Further embodiments within the scope of this invention may include more than two annular “open cell” spaces or gaps between circumferential arrays 12, 12A etc of loops 14, depending upon the number of cross-links 50 dividing up each annular space between adjacent arrays 12, 12A, . . . 12F. For example, one embodiment may extend the general pattern of open spaces 30 to comprise three annular “open spaces” or gaps 30, each one of which spans about a third of the periphery (about 120 degrees) of the stent assembly 10. In a further embodiment, a varying number (e.g. 2, 3 or more) of cross-links may be disposed between adjacent arrays 12, 12A etc., to provide any particular desired variation in bending and/or in receptability to through-wall penetration by several stent assemblies 10.

After the insertion of a stent assembly 10 in a vessel, bifurcated or otherwise, and upon expansion of the adjacent circumferential loops 14 of each array 12, 12A etc., each of the cross-links 50 between adjacent circumferential arrays 12, 12A, etc. may, in one embodiment, be re-oriented slightly or pivoted, as viewed radially inwardly, indicated by the arrow “P”, in FIG. 1A, so as to be rotated or pivoted from an oblique orientation with respect to its alignment with longitudinal axis “L” of the stent assembly 10, to an orientation which is more parallel to the longitudinal axis “L” of the stent assembly 10. Such a movement of those cross-links 50 assists in forestalling any shortening of the length of the stent assembly 10 as it expands within the vasculature of a patient. Such annular or circumferential disposition of the semi-circumferential gaps or spacings 30 during expansion of the stent assembly 10, and the rotation of the cross-links 50 however, remain in general circumferential disposed alignment with respect to the longitudinal axis of the stent assembly 10, and not obliquely angled with respect thereto. Such stent assembly 10 foreshortening during expansion thereof can be, however, primarily prevented by the expansive common circumferential and longitudinally directed deformation of the curves or bends 14 due to their unique curvilinear configuration, which comprises the structure being moved radially outwardly.

The minimal number of cross-links 50 between longitudinally adjacent circumferential arrays 12, 12A, . . . , 12F of loops 14 adds to the stent assembly's flexibility and adaptability of that stent assembly 10 in the curved vasculature of a patient. Similarly, the untethered adjacent bends 14 in the respective circumferential arrays 12, 12A, . . . , 12F allows for substantially uniform radial strength over the length of the stent assembly 10 permitting substantially uniform expansion and avoidance of such effects as “dog boning” or the foreshortening of that stent assembly 10 within a patient. In an embodiment, each of the cross-links 50 extending from a circumferential array 12A, 12B, . . . , 12F, is substantially circumferentially offset from each cross-link 50 extending from the same circumferential array on its longitudinally opposite side, thus providing flexibility and adaptability of that stent assembly 10 in the curved vasculature of a patient. In an embodiment, the circumferential offset is about 90 degrees as shown by circumferential offsets 54 between cross-links 50.

In an aspect of the invention, a method is provided for the extension of a first stent assembly with a second stent assembly by overlapping a portion of the longitudinal ends (e.g. first end 16 or second end 32 as shown in FIG. 1A) of stent assemblies in accordance with the strut design of the present invention, to create an arrangement readily known to those of ordinary skill in the art as “kissing stents.” A first stent assembly 10 is inserted and expanded into a vessel. A second stent assembly 10 is then inserted through the longitudinal opening of the first stent assembly so that it partially overlaps a longitudinal section of the first stent assembly 10, after which the second assembly is expanded in place. The second stent assembly can be of a smaller initial diameter to better accommodate fitting within the first stent assembly 10 and/or for simultaneous deployment/expansion (wherein the stents are initially overlapping and are inserted together). A minimal amount of strut structure embodied in each stent assembly of the present invention reduces the likelihood of interaction with tissue material along the overlapping portions of their outer circumferences.

The thicknesses of the struts can be optimized to promote flexibility, minimal surface contact, and the expansiveness of the spaces between struts. In an embodiment, the struts are of a thickness of between about 60 and 100 microns and, at non-connecting joints, can average about 80 microns in width which can, for example, be suitable for medium sized vessels (from 3 mm to less than 4 mm in diameter). In another embodiment, the struts are of a thickness of between about 50 and 80 microns and, at non-connecting joints, average about 65 microns in width which can, for example, be suitable for smaller sized vessels (less than 3 mm in diameter). In another embodiment, the struts are of a thickness of between about 110 and 150 microns and, at non-connecting joints, can average about 130 microns in width which can, for example, be suitable for larger sized vessels (4 mm in diameter and larger).

Loops or curves 14 are shown in an enlarged representation in FIG. 1B in an embodiment. In an embodiment, the arcuately-shaped hairpin-like curves 14 have a smoothly-curved concave side 17 and a smoothly-curved convex side 19. Thus, the concave and convex sides 17 and 19 are correspondingly curved circumferentially, that is, curved in a similar direction.

The direction of loops or curves 14 substantially reverse through bends 60 and 62 in a switchback or hairpin-like manner. Bend 60 includes a region 20 and bend 62 includes a region 27, wherein region 20, 27 has, in an unexpanded state, relatively tighter degrees of curvature, and smaller radii “r” extending from corresponding centers of curvature “c” relative to the bends 60, 62, than those of other regions of the stent. The regions about strut connection points such as, for example, region 25 about mid-portion 52, can also have generally tighter curvatures than, for example, the smoothly-curved concave side 17 of the hairpin-like curves 14. In an embodiment, a minimum radius of curvature “r” of each bend along the entire surface of the unexpanded stent, that is, not expanded beyond a point appropriate prior to deployment, is about 65 microns. In an embodiment, the minimum radius of curvature is about 80 microns. In an embodiment, the stent has one or more layers of coating material while having a minimum radius of curvature of about 50 microns.

The relatively large minimum radius of curvature provides a highly favorable surface over which coating materials can be deposited. Distributing curvature more evenly over the entire stent helps avoid the inclusion of regions of tight curvature, which is a key disadvantage of prior designs. For example, the overall openness of the curves 14 helps avoid a structural blockage that could prevent a consistent coating over the entire stent surface. On the other hand, a region having a small radius of curvature, for example, less than 65 microns, may more likely receive less material than other regions having larger radii of curvature, resulting in an insufficient coating about the surface corresponding to the region having the smaller radius of curvature.

An inconsistent coating process may prompt thicker layers of material to be applied overall to the stent surface in order to ensure adequate coverage overall. Thicker layers of material, particularly metallic material, can detrimentally affect biomechanical properties of the stent, including flexibility and tissue-to-stent surface contact. In addition, the areas of relatively low curvature help avoid the effect of “webbing,” wherein a region having a tight curvature acting as a crevice can essentially be filled in and could cause the coating to stretch apart and/or split during expansion of the stent, including the area of tight curvature. Moreover, regions of tight curvature (with or without coatings) can be subject to greater mechanical stresses when they are opened (such as during expansion), thus increasing the likelihood of metal fatigue, fractures, and/or increased post-expansion recoil.

In an embodiment, opposing surfaces or portions of the stent, i.e., where a straight line that is normal relative to one location on the external surface of the stent can extend outwardly to another location on the external surface of the stent, are separated by a minimal distance and can help avoid such issues with various coating processes including, for example, electro-magnetic interference, uneven coating, webbing, and/or cracking. In an embodiment, all opposing surfaces of embodiments of the stent structure previously described are separated by normal straight-line distances (or spans) by a minimum of about 130 microns. Referring to FIG. 1B, exemplary straight-line normal spans 70 between opposing strut surfaces, or portions, are of at least this distance. In another embodiment, all normal straight-line distances or spans of opposing surfaces (e.g. normal spans 70) are a minimum of about 160 microns.

The “open” curvature and/or substantially non-interfering characteristics of various embodiments of the invention promote a structure conducive to various coating technologies including, in particular, those involving streams of coating particles and/or bombarding particles directed at the surface of an embodiment (e.g. the struts of annular arrays 12, 12A, . . . , 12F and cross-links 50) of the stent structure. In an embodiment, the coating process comprises directing a stream of particles (e.g. coating and/or bombarding atoms or ions) toward the stent structure. In an embodiment, a coating process comprising at least one of electrochemical deposition, chemical-vapor deposition, electroplating, electro-polishing, ion-assisted deposition, and/or ultrasonic spraying.

In an embodiment, the struts are layered with inert biocompatible materials, including gold, silver, platinum, and/or various non-metallic materials.

In an embodiment, one or more layers is provided with ion-assisted deposition onto the stent structure, such as, for example, through methods which use one or more magnetrons such as described in pending U.S. patent application Ser. No. 09/999,349 by Sahagian, published Sep. 26, 2006 as United States Patent Application Publication No. 2002/0138130A1 and pending U.S. patent application Ser. No. 11/843,376, published as United States Publication No. 2008-0177371-A1, entitled “IMPLANTALE DEVICES AND METHODS OF FORMING THE SAME” and U.S. patent application Ser. No. 11/843,402, published as United States Publication No. 2008-0215132-A1, entitled “IMPLANTABLE DEVICES HAVING TEXTURED SURFACES AND METHODS OF FORMING THE SAME”, each by S. Eric Ryan and Richard Sahagian, and each filed on Aug. 22, 2007, the contents of each of which are herein incorporated by reference in their entirety. Various embodiments of these apparatus and methods involve actively and/or passively biasing a substrate with electrical charge and thus increasing the attraction of charged coating and/or bombarding atoms or ions, for which various embodiments of the present invention can help improve the uniformity of the magnetic attraction.

In an embodiment, the struts of annular arrays 12, 12A, . . . , 12F and cross-links 50 are comprised of a highly radiopaque substrate such as, for example, cobalt-chromium material, stainless-steel, and nitinol material. In a further embodiment, such as in accordance with previously cited and incorporated U.S. patent application Ser. No. 11/843,376, published as United States Publication No. 2008-0177371-A1, gradations of platinum and palladium ions are implanted onto a cobalt chromium base through variations of these methods to produce an adhesion layer of essentially palladium or gold, a transition layer of increasing platinum content and decreasing palladium content and a bio-compatible metal capping layer of essentially platinum or having, at least, a predominance of platinum. In an embodiment, the metal capping layer consists essentially of pure platinum. In further embodiments, the palladium and platinum layers can be from about 100 angstroms and up to about 5,000 angstroms thick, preferably greater than for example, about 500 angstroms thick, and less than about 2,500 angstroms, such that they are optimized to maximize the smoothness and stability of the layers. The thicknesses may depend upon various parameters, including the size and projected expansion of the stent assembly.

In an embodiment the metal capping layer is manufactured with at least one of platinum, platinum-iridium, tantalum, titanium, tin, indium, palladium, gold and alloys thereof. In an embodiment, the metal capping layer and all layers within the metal capping layer (such as, for example, an adhesion layer, or no layers between the substrate and metal capping layer) have a combined thickness of less than about a micron. In an embodiment, the metal capping layer and all layers within the metal capping layer have a combined thickness of less than about 0.5 microns. In an embodiment, the metal capping layer and all layers within the metal capping layer have a combined thickness of less than about 0.25 microns.

In an embodiment, surface modifications are applied to struts of annular arrays 12, 12A, . . . , 12F and cross-links 50 that provide textured surfaces such as in accordance with previously cited and incorporated U.S. patent application Ser. No. 11/843,402, published as United States Publication No. 2008-0215132-A1. The texturing can improve the surface of the stent for purposes of encouraging healthy endothelial growth upon deployment, providing a more adhesive surface for additional layers such as polymers having drug-eluting properties, and/or improving the retention and avoiding undesired slippage between the surface of the stent and a delivery system (e.g. a balloon catheter) during delivery.

In various embodiments of the invention, one or more of the magnetrons 100 of the apparatus of FIG. 3 can be employed to perform ion-assisted deposition of the previously disclosed applicable coatings such as, for example, the gradated adhesion and biocompatible metal capping layers, and textured surfaces in accordance with previously cited U.S. application Ser. Nos. 11/843,376 and 11/843,402. In an embodiment, an apparatus 80 is provided for processing multiple stents in accordance with the invention using a batch process with one or more magnetrons providing magnetic fields 130. A fixture 91 holding a stent 10 between magnetrons 100 is attached at one end to a wheel 90 which is rotatable and driven via an axle 97 and an actuating mechanism (not shown). After one stent 10 has been coated by magnetrons 100, another stent 10 attached to wheel 90 can be actuated into place between magnetrons 100. In an embodiment, numerous stents 10 can be similarly attached to wheel 90 and coated in an automated manner. Wheel 90 and attached stents 10 and magnetrons 100 are contained in a vacuum chamber 82. A vacuum can be drawn from chamber 82 using a vacuum pump 88. Vacuum pumping may thereafter be throttled by a valve 83 and a noble gas, for instance, argon or xenon, may be introduced from a source 84 through a port 85 into chamber 82.

The chamber 82 may continue to be filled with the noble gas in order to generate ions for the purpose of impacting the surface of stent 10 during cleaning and/or co-deposition of coating materials such as those previously described. In an embodiment, an electrical bias may be applied to stent 10 such as, for example, between about −20VDC and −1000VDC, to attract bombarding ions and/or coating materials. In an embodiment, a relatively strong bias, for example, between about −200VDC and −1000VDC, can be applied for attracting bombarding particles such as noble ions, which can be used for cleaning. In an embodiment, a relatively lower bias, for example, between about −20VDC and −100VDC, can be applied for deposition of coating particles and attracting co-deposited noble ions. The previously disclosed geometries of stents in accordance with various embodiments of the invention such as, for example, stent 10, can promote a relatively more even bombardment of the noble ions. As discussed above, the relatively more open geometries of various embodiment of the invention can help avoid blockage of the impacting ions and promote a more uniform magnetic attraction provided by the applied electrical charge.

Various coating technologies implementing streaming particles and/or electro-magnetic biasing can also be improved by the stent geometry such as electrochemical deposition, chemical-vapor deposition, electroplating, electro-polishing, ion-assisted deposition, and/or ultrasonic spraying. The spraying of polymers such as those with active therapeutic agents, for example, or other polymers known to those of ordinary skill in the art, can be applied in common coating applications and can benefit from the improved geometric designs in accordance with various embodiments of the invention.

While various embodiments of the invention can promote an optimal coating surface, the strut pattern can also provide excellent biomechanical properties that promote even expansion, strong radial force, minimal tissue-to-stent contact, avoidance of foreshortening, and avoidance of dog-boning, among other biomechanical properties.

It will be understood by those with knowledge in related fields that the use of alternate or varied materials and modifications to the methods disclosed herein are apparent. This disclosure is intended to cover these and other variations, uses, or other departures from the specific embodiments as come within the art to which the invention pertains.

1. A flexible, expandable, elongated stent assembly comprising:
a generally cylindrically-shaped channel that extends along a longitudinal axis; and
a plurality of openings in the channel, said openings being defined by a structure of connected circumferential arrays of webs or bends, wherein, in an unexpanded state of the stent assembly, the webs or bends and their connections have minimum radii of curvature of at least about 65 microns.

2. The flexible, expandable stent assembly as recited in claim 1, wherein the webs or bends and their connections have minimum radii of curvature of at least about 80 microns.

3. (canceled)

4. The flexible, expandable, elongated stent assembly of claim 1, wherein said webs or bends are in a switchback configuration and the circumferential arrays are connected to one another by a plurality of cross-links and, wherein, from a flattened radially-directed view, each and every web, bend and cross-link of the stent assembly forms a path of an arc.

5. The flexible, expandable stent assembly as recited in claim 4, wherein a substantial portion of each and every web, bend, or cross-link forms an arc of the same concavity with respect to the circumference of said stent assembly.

6.-10. (canceled)

11. The flexible, expandable, elongated stent assembly of claim 1, wherein said assembly has a substrate with a surface and one or more surface layers the surface of the substrate.

12. The flexible, expandable, elongated stent assembly of claim 11, wherein said one or more surface layers comprises a metal capping layer comprising a predominant proportion of a substantially biocompatible metal.

13. (canceled)

14. The flexible, expandable, elongated stent assembly of claim 12, wherein said metal capping layer consists essentially of pure platinum.

15. The flexible, expandable, elongated stent assembly of claim 12, wherein said one or more surface layers further comprises an adhesion layer comprising a portion including at least 50% palladium directly on the surface of the substrate, the adhesion layer positioned between the substrate and said metal capping layer.

16. The flexible, expandable, elongated stent assembly of claim 12, wherein the metal capping layer and all surface layers within the metal capping layer have a combined thickness of less than or equal to about 0.5 microns.

17. The flexible, expandable, elongated stent assembly of claim 16, wherein metal capping layer and all surface layers within the metal capping layer have a combined thickness of less than about 0.25 microns.

18. (canceled)

19. The flexible, expandable, elongated stent assembly of claim 11 wherein at least one of said surface layers have a density of greater than about 95% full bulk density.

20. The flexible, expandable, elongated stent assembly of claim 1, wherein external surfaces of the webs or bends and cross-links are separated from opposing external surfaces of the webs or bends along normal straight-line spans by a minimum of about 130 microns.

21.-27. (canceled)

28. A flexible, expandable, elongated stent assembly comprising:
a substantially cylindrical channel that extends a longitudinal axis;
a plurality of openings in the channel, said openings being defined by a substrate structure of substantially smoothly and arcuately-shaped webs or bends, wherein external surfaces of the webs or bends are separated from opposing external surfaces of the webs or bends along normal straight-line paths by at least about 130 microns; and
one or more surface layers on said webs or bends.

29. The flexible, expandable, elongated stent assembly of claim 28 wherein external surfaces of said webs or bends are separated from opposing external surfaces of said webs or bends along normal straight-line paths by at least about 160 microns.

30. (canceled)

31. A flexible, expandable, elongated stent assembly comprising:
a generally cylindrically-shaped channel that extends along a longitudinal axis;
a plurality of openings in the channel, said openings being defined by a structure of connected circumferential arrays of webs or bends, wherein, in an unexpanded state of the stent assembly, the webs or bends and their connections have minimum radii of curvature of greater than about 50 microns; and
one or more surface layers on the stent assembly.

32.-34. (canceled)

35. A method of coating a flexible, expandable stent assembly, said method comprising:
providing a stent comprising a generally cylindrically-shaped channel that extends along a longitudinal axis, and having a plurality of openings therein, said openings being defined by a substrate structure of webs or bends, wherein, in an unexpanded state of the stent assembly, the webs or bends have minimum radii of curvature of at least about 65 microns; and
directing at least one stream of coating particles toward the substrate structure so as to form one or more layers of coating particles over the substrate structure.

36. The method of claim 35, wherein the webs or bends have minimum radii of curvature at least about 80 microns.

37. The method of claim 35, wherein the directing at least one stream of coating particles toward the substrate structure comprises the use of at least one of electrochemical deposition, electroplating, electro-polishing, and ion-assisted deposition.

38. The method of claim 37, wherein the step of directing at least one stream of coating particles comprises an ion-assisted deposition process including simultaneously directing the coating particles and bombarding ions toward the substrate structure in a substantially collinear manner.

39. The method of claim 35, wherein the directing at least one stream of coating particles toward the substrate structure comprises forming a metal capping layer over the substrate structure, the metal capping layer comprising a predominant proportion of a highly biocompatible metal.

40. (canceled)

41. The method of claim 39, wherein the biocompatible metal consists essentially of platinum.

42. (canceled)

43. The method of claim 41, wherein the combined thickness of the capping layer and all surface layers within metal capping layer is less than about 0.5 microns.

44. The method of claim 41, wherein the combined thickness of the metal capping layer and all surface layers within the metal capping layer is less than about 0.25 microns

45.-49. (canceled)

50. The method of claim 35, wherein external surfaces of the webs or bends and cross-links are separated from opposing external surfaces of the webs or bends along normal straight-line spans by a minimum of about 130 microns.

51. The method of claim 35, wherein a substantially uniform magnetic field is generated about the webs or bends while the at least one stream of coating particles is directed toward the substrate structure.

52. (canceled)

53. The method of claim 51, wherein a voltage across the webs or bends is actively applied to the webs or bends.

54. The method of claim 51, wherein the voltage across the webs or bends is between about −20VDC and −1000VDC.

55.-64. (canceled)
